(19) United States
(12) Patent Application Publication
(10) Pub. No.: US 2024/0257952 A1
(43) Pub. Date: Aug. 1, 2024

(54) MEDICAL PROCEDURE COST COMPARISON TOOL

(71) Applicant: OPTUM, INC., Minnetonka, MN (US)

(72) Inventor: Seth O'Neal, Arlington, TX (US)

(21) Appl. No.: 18/159,810

(22) Filed: Jan. 26, 2023

(51) Int. Cl. G16H 40/20; G16H 10/60

(52) U.S. Cl. CPC G16H 40/20; G16H 10/60

(57) ABSTRACT

A system and methods for comparing medical procedure costs are disclosed. The method can include receiving a first user input that includes a medical procedure identifier and travel parameters for a user, identifying one or more medical providers that can provide a medical procedure associated with the medical procedure identifier, generating a plurality of candidate procedure packages for the medical procedure by aggregating cost and scheduling information from a plurality of remote sources associated with each of the one or more medical providers and one or more travel-related entities, presenting the plurality of candidate procedure packages to the user via a user interface, receiving a second user input selecting one of the plurality of candidate procedure packages, and scheduling the medical procedure and booking round-trip travel through a respective subset of the plurality of remote sources.

### BACKGROUND

Medical tourism, which generally refers to the act of a person traveling aboard for medical treatment, has been a growing trend in recent years. In some cases, a person may travel for a medical treatment that is not available locally (e.g., in their home country) or for more specialized or advanced care. In other cases, a person may travel for lower-cost medical treatments, shorter wait times, and the like. Whatever the case may be, it can be a difficult and confusing task to plan and schedule a medical-related trip to another country. For example, flights, hotel stays, local transportation, and even the medical procedure itself are generally booked through disparate systems that must be accessed individually (e.g., through corresponding websites), which can be unduly burdensome for potential patients.

### SUMMARY

One implementation of the present disclosure is a method. The method generally includes receiving a first user input that includes a medical procedure identifier and travel parameters for a user, identifying one or more medical providers that can provide a medical procedure associated with the medical procedure identifier, generating a plurality of candidate procedure packages for the medical procedure by aggregating cost and scheduling information from a plurality of remote sources associated with each of the one or more medical providers and one or more travel-related entities, presenting the plurality of candidate procedure packages to the user via a user interface, receiving a second user input selecting one of the plurality of candidate procedure packages, and scheduling the medical procedure and booking round-trip travel through a respective subset of the plurality of remote sources. In some implementations, the one or more travel-related entities are identified based on the travel parameters. In some implementations, each of the plurality of candidate procedure packages indicates: i) a cost for performing the medical procedure at a medical facility associated with a respective one of the one or more medical providers, and ii) a cost for round-trip travel to/from the medical facility.

Another implementation of the present disclosure is a system. The system generally includes a processor and memory having instructions stored thereon that, when executed by the processor, cause the system to receive a first user input that includes a medical procedure identifier and travel parameters for a user, identify one or more medical providers that can provide a medical procedure associated with the medical procedure identifier, generate a plurality of candidate procedure packages for the medical procedure by aggregating cost and scheduling information from a plurality of remote sources associated with each of the one or more medical providers and one or more travel-related entities, present the plurality of candidate procedure packages to the user via a user interface, receive a second user input selecting one of the plurality of candidate procedure packages, and schedule the medical procedure and booking round-trip travel through a respective subset of the plurality of remote sources. In some implementations, the one or more travel-related entities are identified based on the travel parameters. In some implementations, each of the plurality of candidate procedure packages indicates: i) a cost for performing the medical procedure at a medical facility associated with a respective one of the one or more medical providers, and ii) a cost for round-trip travel to/from the medical facility.

Yet another implementation of the present disclosure is a non-transitory computer readable medium having instructions stored thereon that, when executed by a processor, cause a device to receive a first user input that includes a medical procedure identifier and travel parameters for a user, identify one or more medical providers that can provide a medical procedure associated with the medical procedure identifier, generate a plurality of candidate procedure packages for the medical procedure by aggregating cost and scheduling information from a plurality of remote sources associated with each of the one or more medical providers and one or more travel-related entities, present the plurality of candidate procedure packages to the user via a user interface, receive a second user input selecting one of the plurality of candidate procedure packages, and schedule the medical procedure and booking round-trip travel through a respective subset of the plurality of remote sources. In some implementations, the one or more travel-related entities are identified based on the travel parameters. In some implementations, each of the plurality of candidate procedure packages indicates: i) a cost for performing the medical procedure at a medical facility associated with a respective one of the one or more medical providers, and ii) a cost for round-trip travel to/from the medical facility.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

FIG. 1 is a block diagram of a communication structure for comparing medical procedure costs, according to some implementations.

FIG. 2 is a detailed block diagram of a medical procedure cost comparison tool, according to some implementations.

FIG. 3 is a flow diagram of a process for comparing medical procedure costs, according to some implementations.

FIGS. 4-6 are various example user interfaces for comparing medical procedure costs, according to some implementations.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Referring generally to the figures, a medical procedure cost comparison tool is shown, accordingly to various implementations. As described herein, the medical procedure cost comparison tool is a system that aggregates cost and scheduling data for medical procedures at disparate locations (e.g., different countries). Moreover, the medical procedure cost comparison tool can plan travel to and from medical facilities, pre- and post-care events, hotel stays, etc., and can aggregate travel-related cost and scheduling data. Generally, the medical procedure cost comparison tool generates multiple “candidate procedure packages” based on the aggregated cost and scheduling data, which can then be presented to a user. Responsive to a user selection of a candidate procedure package, the medical procedure cost comparison tool may be configured to schedule the medical procedure, travel, etc., and can even adjudicate payments to various companies or vendors (e.g., hotels, airlines, etc.) associated with the selected package.

Consider, for example, a patient that needs a total knee replacement. As it will be appreciated, directly comparing costs for having total knee replacement surgery domestically (e.g., within the United States) and internationally may not be accurate, as traveling internationally for surgery would incur various travel-related expenses (e.g., airfare) which are not always applicable when receiving treatment locally. Further, as mentioned above, it can be time consuming and confusing to search for cost information for multiple different medical providers, airlines, hospitality companies, etc.; not to mention the time and effort required to coordinate scheduling, book the medical procedure and travel, and the like. To this point, the medical procedure cost comparison tool described herein can greatly reduce the time and complexity typically associated with scheduling medical procedures international and provides an easy-to-use interface for users to compare medical costs (e.g., both domestically and internationally). Continuing the example above, the medical procedure cost comparison tool can present the patient a plurality of candidate procedure packages that indicate a more representative, total cost for the associated medical procedure—including travel expenses.

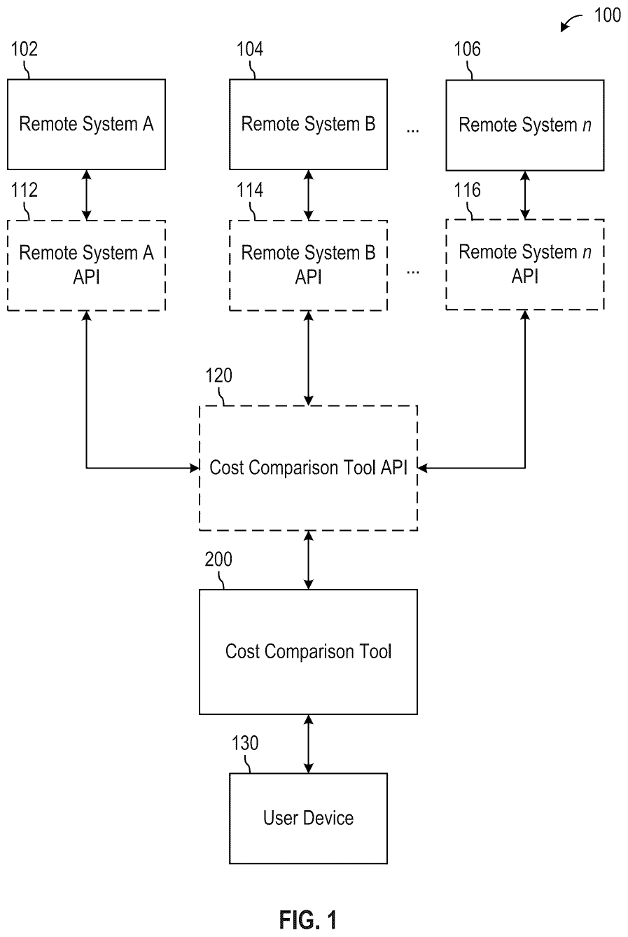

Turning first to FIG. 1, a block diagram of a communication structure 100 for comparing medical procedure costs is shown, according to some implementations. The aforementioned medical procedure cost comparison tool is illustrated in FIG. 1 as a cost comparison tool 200 which generally aggregates cost and scheduling information from a plurality of remote sources to generate candidate procedure packages for a user. The remote sources, shown as remote systems 102-106, can generally include any remote computing system that maintains cost and/or scheduling data for an associated entity (e.g., a business, a medical facility, a medical provider, etc.). For example, remote systems 102-106 can be servers (e.g., cloud or web servers), workstations, or even laptop computers that maintain cost and scheduling data for associated medical facilities, health care providers, hospitality organizations (e.g., hotels), airlines, and the like. While only three remote systems are shown, it should be appreciated that communication structure 100 can generally include any number of remote systems (e.g., n remote systems, where n is any positive integer).

As described herein, “cost” information is generally a cost or price of a service or event. For example, cost information for a medical provider generally includes the cost for receiving a particular medical treatment. In some implementations, medical procedure cost information also includes pre- and post-care events (e.g., surgical consultations, rehabilitation, post-care checkups, etc.). As another example, cost information for travel-related entities can include ticket prices, fees for hotel stays, rental vehicle costs, and the like. “Schedule” information generally refers to information relating to scheduling or booking an event or service. For example, scheduling information for a medical provider may include available dates and/or times for performing a medical procedure, an amount of time (e.g., days, hours) to perform the procedure, days and times for pre- and post-care events, etc. Scheduling information for travel-related entities can include mass transportation (e.g., flights, train rides, etc.) availability (e.g., days and times), rental car availability, shuttle availability, hotel availability, etc. For example, scheduling information for an airline may include available tickets for round-trip airfare to/from a medical provider, where the available tickets indicate a date and time of the flight.

As shown, in some implementations, remote systems 102-106 each optionally include a corresponding application programming interface (API), shown as remote system APIs 112-116, that facilitates the communication of data to/from an associated one of remote systems 102-106. For example, remote system 102 (“Remote System A”) may include remote system API 112 (“Remote System A API”) which facilitates communication between remote system 102 and any external systems, including cost comparison tool 200. Because each of remote systems 102-106 may be different, it should be appreciated that each of remote system APIs 112-116 may be any suitable API. For example, one or more of remote system APIs 112-116 may be REST APIs, ROA APIs, etc. In some implementations, cost comparison tool 200 itself also includes an API, shown as cost comparison tool API 120. Like remote system APIs 112-116, cost comparison tool API 120 facilitates communication with cost comparison tool 200, and may be any suitable API (e.g., a RESTful API). It should also be noted that, in various other implementations, remote systems 102-106 and cost comparison tool 200 may communicate directly (e.g., without the use of APIs).

In some implementations, communication structure 100 includes a user device 130 that a user utilizes to communicate with and/or control cost comparison tool 200. In some such implementations, user device 130 and cost comparison tool 200 may communicate directly. In other implementations, user device 130 communicates with cost comparison tool 200 via cost comparison tool API 120. User device 130 is generally any computing device that allows a user to provide data to and/or receive data from cost comparison tool 200. For example, user device 130 may be a smartphone, a personal computer (e.g., a laptop or desktop computer), a server, a workstation, or the like. In some implementations, user device 130 includes at least a display screen (e.g., an LED or LCD screen) for displaying data relating to cost comparison tool 200 and a user input device, such as a mouse, a keyboard, a number pad, a joystick, etc. For example, user device 130 may include a touch screen that allows a user to both view and input data. To this point, user device 130 is generally how a user interacts with cost comparison tool 200 to retrieve cost and scheduling information.

To better understand how the components of communication structure 100 interact, an example use-case is provided herein. Consider, for example, the previous example of a patient that is comparing costs for total knee replacement surgery. In this scenario, the patient may utilize user device 130 (e.g., their smartphone) to enter parameters for a cost comparison. For example, the user of user device 130 may enter the type of procedure to be performed (e.g., knee replacement surgery), their location (e.g., the patient's home address), their insurance information, desired travel and/or procedure dates, and the like. In some cases, the user may also enter travel related information such as whether they are willing to fly, preferred airlines, preferred hotels, whether they have a valid passport, etc. Various other parameters may also be entered, which are described in greater detail below.

User device 130 may communicate these user inputs to cost comparison tool 200, which then requests or retrieves associated information from each of remote systems 102-106. For example, cost comparison tool 200 may first identify medical providers that perform knee replacement surgery and may then determine which identified medical providers have scheduling availability (e.g., within a window provided by the patient or within a predetermined time horizon). Generally, the medical providers identified by cost comparison tool 200—and, in fact, any of the entities associated with remote systems 102-106—have previously agreed to sending data to cost comparison tool 200. For example, an entity that operates cost comparison tool 200 may establish agreements with entities associated with each of remote system 102-106 prior to requesting or retrieving cost and scheduling data.

In any case, after identifying a set of medical providers that can perform the desired medical procedure (and optionally within the patient's desired time frame), cost comparison tool 200 may retrieve or request cost and scheduling data from associated ones of remote systems 102-106. Additionally, cost comparison tool 200 may determine travel-related requirements for each medical provider, such as flights to the nearest airport, additional ground transportation to/from the medical facility, hotel stays before and/or after the procedure, etc., and may then request cost and scheduling data from associated travel-related entities. For example, one or more of remote systems 102-106 may be operated by, or maintain data associated with, various airlines, hotels, car rental services, train lines, bus lines, and the like.

As described herein, “retrieving or requesting” cost and scheduling data can include sending an API call (e.g., cost comparison tool 200) to associated remote system APIs 112-116. For example, cost comparison tool 200 may send an API call to any of remote system APIs 112-116 associated with an airline to request cost and scheduling information from the airline's system. In response, the associated system of remote systems 102-106 can return the requested data or a message indicating that the data was not available, the request could not be completed, etc. Alternatively, in some implementations, cost comparison tool 200 may be allowed direct access to data in remote systems 102-106, such that cost comparison tool 200 can directly retrieve cost and scheduling data.

Cost comparison tool 200 may then aggregate the received or retrieved cost and scheduling data to generate multiple candidate procedure packages for the user. In some implementations, the candidate procedure packages are displayed via a user interface of user device 130, such that the user can view the candidate procedure packages and/or select a candidate procedure package for booking. Generally, each candidate procedure package indicated a total cost for receiving the user-defined medical treatment at an associated facility, to include all or most travel-related expenses. For example, a candidate procedure package for total knee replacement at a medical facility in Mexico may include the cost for the procedure, travel costs to/from the medical facility in Mexico, any pre- and/or post-care visit/travel costs, etc. In some implementations, each candidate procedure package may further indicate scheduling information such as a day of the procedure, travel times, a total length (e.g., in days) of the trip to receive treatment, etc.

In some implementations, a user may select a candidate procedure package via user device 130 in order to proceed with booking travel arrangements, scheduling the procedure, etc. In some such implementations, cost comparison tool 200 can communicate with respective ones of remote systems 102-106 to facilitate booking/scheduling the procedure, travel, etc. For example, cost comparison tool 200 may generate and transmit suitable API calls and may receive confirmation data from remote systems 102-106 one the procedure, travel, etc., is booked. In some implementations, cost comparison tool 200 can facilitate or “adjudicate” payment for all expenses relating to a selected candidate procedure package. For example, cost comparison tool 200 can prompt a user to input (e.g., via user device 130) payment information (e.g., credit card or bank information) and/or can transmit payments to remote systems 102-106 (e.g., bank accounts associated with each entity relating to the selected candidate procedure package).

In some implementations, cost comparison tool 200 is configured to generate candidate procedure packages for receiving medical treatment domestically, in addition to the aforementioned international candidate procedure packages. For example, cost comparison tool 200 can collect cost and scheduling information for one or more medical providers local to the patient or within a threshold distance of the patient (e.g., within 150 miles, within the United States, etc.). In this way, a user can compare the cost of receiving medical treatment locally as opposed to traveling for the treatment. Additionally, in some implementations, cost comparison tool 200 may be able to account for a user's medical insurance when determine domestic or local treatment costs, to provide for a more direct comparison with international treatment. In some implementations, where cost and/or scheduling information is not directly available, cost comparison tool 200 may be configured to determine an average or estimated cost for a procedure. For example, cost comparison tool 200 may maintain a database of average costs for various medical procedures both domestically and internationally. Alternatively, cost comparison tool 200 may retrieve estimated or average costs from another system or by web scraping. Additional details and features of cost comparison tool 200 are described in greater detail below.

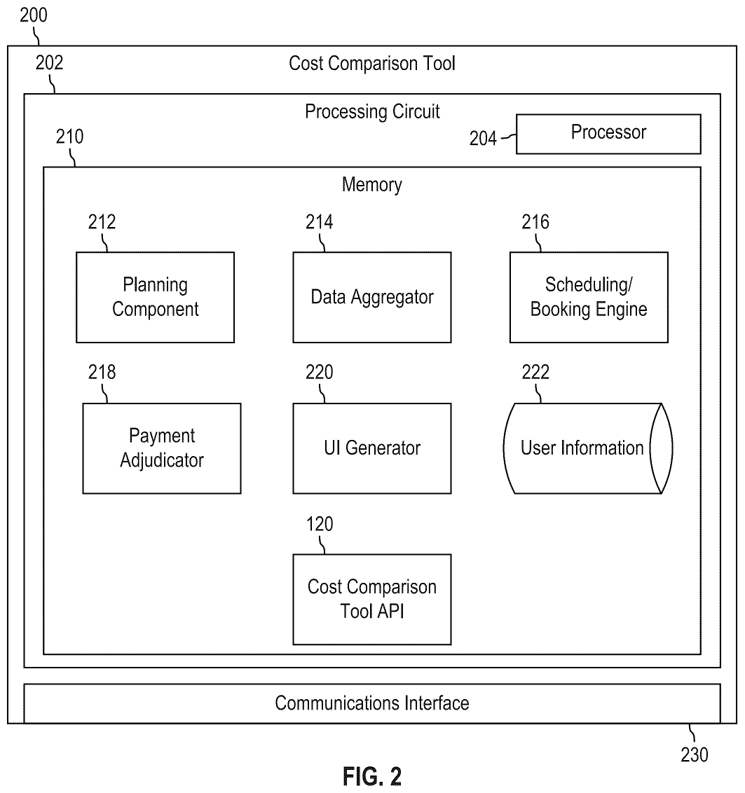

Referring now to FIG. 2, a detailed block diagram of cost comparison tool 200 is shown, according to some implementations. Cost comparison tool 200 is shown to include a processing circuit 202 that includes a processor 204 and a memory 410. Processor 204 can be a general-purpose processor, an application-specific integrated circuit (ASIC), one or more field programmable gate array (FPGAs), a group of processing components, or other suitable electronic processing structures. In some implementations, processor 204 is configured to execute program code stored on memory 210 to cause cost comparison tool 200 to perform one or more operations, as described below in greater detail. It will be appreciated that, in implementations where cost comparison tool 200 is part of another computing device, the components of cost comparison tool 200 may be shared with, or the same as, the host device. For example, if cost comparison tool 200 is implemented via a general purpose computer or server, then cost comparison tool 200 may utilize the processing circuit, processor(s), and/or memory of the general purpose computer or server to perform the functions described herein.

To this point, in some implementations, cost comparison tool 200 is implemented as a cloud service (e.g., hosted on a cloud server) such that cost comparison tool 200 is remotely accessible to various user devices. For example, the various functions of cost comparison tool 200 described herein may be accessed/implemented via a web interface (e.g., a web page), such that a user can interact with cost comparison tool 200 remotely via the Internet. In this manner, cost comparison tool 200 can handle the retrieval and aggregation of immense amounts of data which could typically not be handled by other computing devices, such as smartphones, with lesser processing capabilities. In some implementations, cost comparison tool 200 may be accessed via a software application, such as a smartphone app. In some such implementations, the software application may present front-end user interfaces that a user can interact with to input and/or view information. For example, the software application may communicate with cost comparison tool 200 via the Internet or another type of network (as described in greater detail below). It should therefore be appreciated that cost comparison tool 200, and the functions thereof, can be implemented in a variety of ways; thus, the description provided herein is not intended to be limited solely to the implementation shown in FIG. 2.

Memory 210 can include one or more devices (e.g., memory units, memory devices, storage devices, etc.) for storing data and/or computer code for completing and/or facilitating the various processes described in the present disclosure. In some implementations, memory 210 includes tangible (e.g., non-transitory), computer-readable media that stores code or instructions executable by processor 204. Tangible, computer-readable media refers to any physical media that is capable of providing data that causes cost comparison tool 200 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 210 can include RAM, ROM, hard drive storage, temporary storage, non-volatile memory, flash memory, optical memory, or any other suitable memory for storing software objects and/or computer instructions. Memory 210 can include database components, object code components, script components, or any other type of information structure for supporting the various activities and information structures described in the present disclosure. Memory 210 can be communicably connected to processor 204, such as via processing circuit 202, and can include computer code for executing (e.g., by processor 204) one or more processes described herein.

While shown as individual components, it will be appreciated that processor 204 and/or memory 210 can be implemented using a variety of different types and quantities of processors and memory. For example, processor 204 may represent a single processing device or multiple processing devices. Similarly, memory 210 may represent a single memory device or multiple memory devices. Additionally, in some implementations, cost comparison tool 200 may be implemented within a single computing device (e.g., one server, one housing, etc.). In other implementations, cost comparison tool 200 may be distributed across multiple servers or computers (e.g., that can exist in distributed locations). For example, cost comparison tool 200 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers.

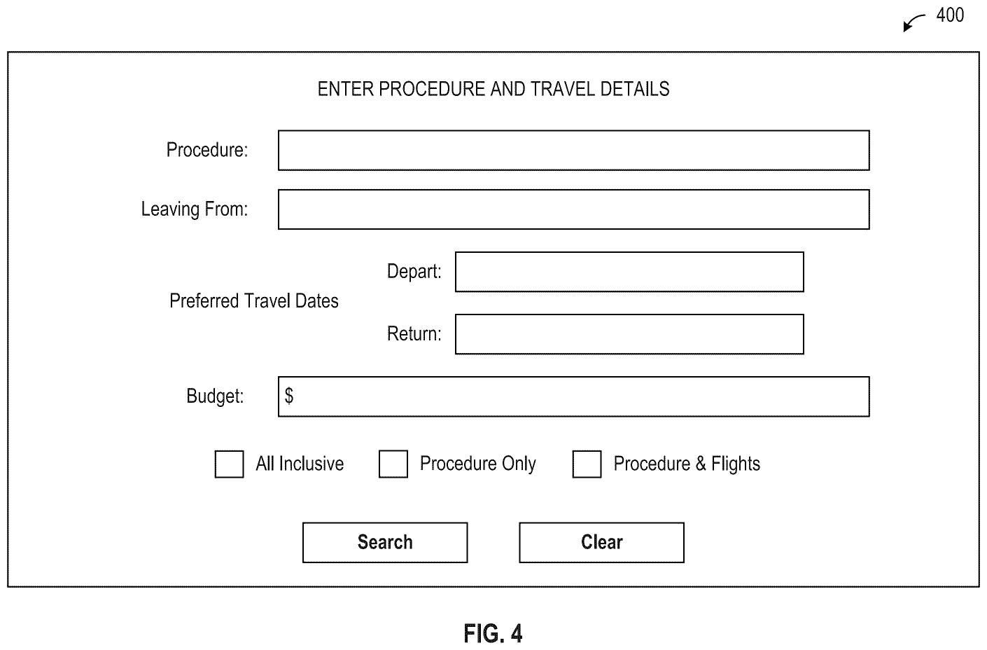

Memory 210 is first shown to include a planning component 212 which receives medical procedure and travel details, identifies one or more medical providers that can provide the medical procedure, and determines, based on the medical procedure and travel details, a travel plan for the user. In some implementations, medical procedure and travel details are received via a user input to a user interface, an example of which is described below with respect to FIG. 4. For example, the user interface may be a web form or fillable fields on a web page that the user can populate with various details relating to the medical procedure to be searched (e.g., the type of medical procedure), and optionally travel-related details. Travel-related details may include, for example, the user's home address, an airport the user would leave from for international travel, preferred travel dates, a budget, whether a rental car and/or hotel are needed, and the like. In some implementations, a user could also enter medical insurance details and passport details. In some implementations, planning component 212 may receive the medical procedure and travel-related details together (e.g., as part of a submitted form), in which case planning component 212 may also be configured to extract the individual medical procedure and travel-related data points for analysis.

Upon receiving and/or extracting the medical procedure and travel-related information, planning component 212 may be configured to identify one or more medical providers that can provide the medical procedure. For example, if the medical procedure is an appendectomy, planning component 212 may identify medical providers that have the capability to, or have agreed to, perform appendectomies. In some implementations, planning component 212 may query a database of medical providers to identify those medical providers that provide the user-defined medical procedure. For example, the database of medical providers may be generated based on agreements between the medical providers and an entity that operates cost comparison tool 200. In some implementations, planning component 212 may transmit a query (e.g., via API call) to remote systems associated with one or more medical providers (e.g., remote systems 102-106) to determine whether each of the one or more medical providers provides the medical procedure. For example, the query may be pseudo-formatted as “do you preform appendectomies?”, in which case each medical provider may return a yes or no answer. It should be appreciated that the methods of identify medical providers provided herein are not intended to be limiting, as planning component 212 component may identify medical providers using other suitable methods.

After identifying medical providers that provide the medical procedure, planning component 212 may determine whether travel is required to reach a medical facility associated with the medical provider. For example, planning component 212 may determine a distance between the user's home address or current location (e.g., determined automatically from the user's device location) and each of the identified medical providers. If the distance between the user and a medical provider—or, more specifically, the medical facility that the medical provider operates out of—is greater than a threshold distance (e.g., 100 miles), then planning component 212 may determine a form of mass transportation that is suitable to reach the medical provider. For example, medical providers that are between 100 and 300 miles of the user may be reached by train or bus, while medical providers farther than 300 miles may require a flight.

In some implementations, planning component 212 further determines whether a lodging is required for the medical procedure. For example, planning component 212 may determine whether the user will need to stay at a hotel or other hospitality facility before or after the procedure. This determination may be made based, at least in part, on the location of each medical provider (e.g., or a medical facility associated with each medical provider), scheduling information of each medical provider, and scheduling information for any transportation companies to/from the medical provider. In some implementations, scheduling information is collected by a data aggregator 214, described in greater detail below. Planning component 212 may determine, for example, a travel plan to each of the medical providers, which can account for dates and/or times of flights and other transportation methods and a date/time of the medical procedure. Thus, planning component 212 can determine whether the user will arrive early or be required to stay after the procedure, in which case a hotel may be required.

Say, for example, that the user is travelling to a medical facility in Italy for a hip replacement and will arrive two days before the medical procedure is scheduled. In this example, planning component 212 may determine that a hotel is required prior to the procedure. Similarly, planning component 212 may determine (e.g., based on medical procedure data) that the user will not be able to fly for two weeks after the surgery; however, the user may not necessarily be required to stay in the hospital for the duration of recovery, so a hotel may be required after the procedure. In some implementations, planning component 212 may also determine, based on medical procedure data, whether the user will be required to attend any pre-care or post-care visits, such as a surgical consultation and/or rehabilitation. Planning component 212 may be configured to account for these pre- and post-procedure events when generating an itinerary. For example, planning component 212 may determine whether additional travel is required for pre- or post-care events, whether a stay at a rehabilitation center is required, etc.

Data aggregator 214 is generally configured to aggregate cost and scheduling data from a plurality of remote sources (e.g., remote systems 102-106). For example, data aggregator 214 may collect cost and scheduling data from medical providers, medical facilities, transportation companies (e.g., airlines, bus and train lines, etc.), hospitality companies (e.g., hotels, motels, etc.), and the like. In some implementations, data aggregator 214 operates in conjunction with planning component 212. Specifically, data aggregator 214 may collect cost and scheduling data responsive to the determinations made by planning component 212 as discussed above. For example, data aggregator 214 may collect cost and scheduling data from those medical providers identified by planning component 212 for a particular medical procedure. Additionally, data aggregator 214 may collect cost and scheduling data from airlines, hotels, etc., based on the travel plan/itinerary prepared by planning component 212.

In some implementations, data aggregator 214 may query remote systems (e.g., remote systems 102-106) associated each medical provider (e.g., each medical provider's internal computer system or server) to request cost and scheduling data. For example, data aggregator 214 may transmit a query (e.g., via API call) that identifies the medical procedure and requests associated cost information and a schedule (e.g., days and/or times) of when the medical procedure could be performed by the medical provider. In some implementations, the returned scheduling data can be evaluated by data aggregator 214 (and/or planning component 212) to determine whether the associated medical provider can perform the medical procedure within a user's designated time frame. For example, the user may indicate that they want the procedure performed within a month; thus, data aggregator 214 may filter out any medical providers that cannot perform the procedure within the timeframe. As another example, some medical providers may simply indicate that they do not have capacity to perform the medical procedure, in which case data aggregator 214 may also filter out any medical providers that cannot perform the procedure at all.

If mass transportation is required, data aggregator 214 may then query one or more remote systems (e.g., remote systems 102-106) associated different transportation providers (e.g., airlines, bus and train lines, etc.) to retrieve cost and scheduling information. For example, data aggregator 214 may retrieve cost and scheduling information from multiple different airlines for flights to/from the medical facility of each identified medical provider. Generally, this will include retrieving cost and scheduling information from multiple different transportation companies. In some implementations, data aggregator 214 first identifies one or more transportation entities (e.g., airlines) that provided round-trip transportation to within a threshold distance of each medical provider, and then requests or retrieves cost and scheduling information. In some implementations, planning component 212 can use the information collected by data aggregator 214 to determine whether additional ground or “last-mile” transportation is needed (e.g., from an airport to a medical facility), in which case data aggregator 214 can also collect cost and scheduling information for rental cars, buses, shuttles, etc. Data aggregator 214 can also query one or more remote systems (e.g., remote systems 102-106) associated different hospitality companies to retrieve cost and scheduling information for hotel stays, rehabilitation, etc.

After collecting cost and scheduling information for all of the identifier medical providers, transportation entities, hospitality entities, etc., data aggregator 214 may generate a plurality of candidate procedure packages. As mentioned above, a candidate procedure package generally indicates a total cost for the medical procedure and round-trip travel, along with any miscellaneous travel-related expenses. For example, an international candidate procedure package may indicate a total cost for the medical procedure at an international medical facility, round-trip airfare, a hotel stay, rehabilitation expenses, bus or train fairs, etc. Data aggregator 214 generally generates multiple candidate procedure packages for multiple different destinations. Candidate procedure packages are also generally created based on the returned scheduling data for each associated entity. For example, data aggregator 214 generally considers the scheduling data for the medical provider, transportation entities, etc., to generate candidate procedure packages, such that the scheduling of the procedure, travel, etc., aligns to minimize the length of the trip.

As described herein, data aggregator 214 can obtain cost and scheduling information in a number of ways. For example, in some implementations, data aggregator 214 send API calls to remote systems 102-106 to request cost and scheduling data. In some implementations, data aggregator 214 accesses a database of cost and/or scheduling data. For example, the database may maintain current or average cost data for various entities (e.g., medical providers, airlines, etc.). In some such implementations, data aggregator 214 may retrieve average cost data from the database when current cost information is not available. For example, the database may be regularly updated with cost information based on feedback from other users of cost comparison tool 200 in order to generate average procedure costs, average travel costs, etc. In some implementations, cost comparison tool 200 maintains a database of average costs based on previous user interactions.

In some implementations, memory 210 includes a scheduling/booking engine 216 which facilitates the scheduling of the medical procedure with a medical provider. In addition, scheduling/booking engine 216 can book travel-related events, such as airfare, hotel stays, etc. For example, a user can be presented with a plurality of candidate procedure packages (e.g., generated by data aggregator 214) and may select a single candidate procedure package for booking. In some such implementations, scheduling/booking engine 216 may schedule and/or book each element of the candidate procedure package (e.g., the procedure, round-trip travel, etc.). In some implementations, scheduling/booking engine 216 automatically schedules/books each element my transmitting API calls or requests to remote systems 102-106, associated with each entity. For example, scheduling/booking engine 216 can transmit booking requests to systems associated with the medical provider, an airline, etc. The request (e.g., API call) may include information relating to the user, such as the user's name, address, demographic information (e.g., age, gender, etc.), and any other necessary information. In this way, each remote system can receive the request and can use the included information to book the corresponding event (e.g., the procedure, travel, etc.).

In some implementations, scheduling/booking engine 216 utilizes artificial intelligence (AI) to call, text, or email various entities for booking a corresponding portion of the candidate procedure package. For example, scheduling/booking engine 216 can use an AI that places phone calls and communicates via speech to book flights, hotels, etc. In some implementations, the AI includes a natural language processor (NLP) that can interpret responses from a human representative of the entity that is called. In some implementations, agreements are in place with each entity prior to cost comparison tool 200 generating the candidate procedure packages, such that scheduling/booking engine 216 may be able to directly book/schedule the procedure, travel, etc. For example, scheduling/booking engine 216 may be able to access each entity's scheduling system (e.g., via remote systems 102-106) to book/schedule an event without outside assistance.

In some implementations, memory 210 includes a payment adjudicator 218 which facilitates payment to one or more entities associated with a selected and booked candidate procedure package. Specifically, payment adjudicator 218 may be configured to sent payments to each entity, such as the medical provider, an airline, etc. In some implementations, payment adjudicator 218 can also collect payment information from a user. For example, payment adjudicator 218 may prompt a user to input credit card information prior to booking a candidate procedure package. In some implementations, user payment information is stored in a user information database 222. Additionally, user information database 222 may be able to store other user data, such as the user's name, a photo of the user, links to other user accounts (e.g., social media accounts), the user's address, user characteristics (e.g., age, gender, height, background, weight, etc.), medical insurance information, passport information, and the like. In some implementations, this user information can be automatically accessed when generating or booking candidate procedure packages. In some implementations, user information database 222 can also contain a record of a user's past medical procedures and/or travel.

In some implementations, memory 210 includes a user interface generator 220 that can generate various user interfaces relating to the various functions of cost comparison tool 200 described herein. For example, user interface generator 220 can generate graphical user interfaces (GUIs) for entering search parameters, reviewing candidate procedure packages, and booking a candidate procedure package. In some implementations, these GUIs are presented via a user interface of cost comparison tool 200. In other implementations, these GUIs are presented via a user interface (e.g., display) of another user device, such as user device 130. For example, user interface generator 220 may generate the user interfaces as web pages that are displayed within a web browsers, such that the user can access the GUIs (and thereby interact with cost comparison tool 200) from anywhere via an Internet connection. Example user interfaces generated by user interface generator 220 are described below with respect to FIGS. 4-6.

Cost comparison tool 200 is also shown to include a communications interface 230 that facilitates communications (e.g., transmitting data to and/or receiving date from) between cost comparison tool 200 and any external components or devices, including remote systems 102-106. Accordingly, communications interface 230 can be or can include one or more wired or wireless communications interfaces (e.g., jacks, antennas, transmitters, receivers, transceivers, wire terminals, etc.) for conducting data communications, or a combination of wired and wireless communication interfaces. In some implementations, communications via communications interface 230 are direct (e.g., local wired or wireless communications) or via a network (e.g., a WAN, the Internet, a cellular network, etc.). For example, communications interface 230 may include one or more Ethernet ports for communicably coupling cost comparison tool 200 to a network (e.g., the Internet). In another example, communications interface 230 can include a WiFi transceiver for communicating via a wireless communications network. In yet another example, communications interface 230 may include cellular or mobile phone communications transceivers.

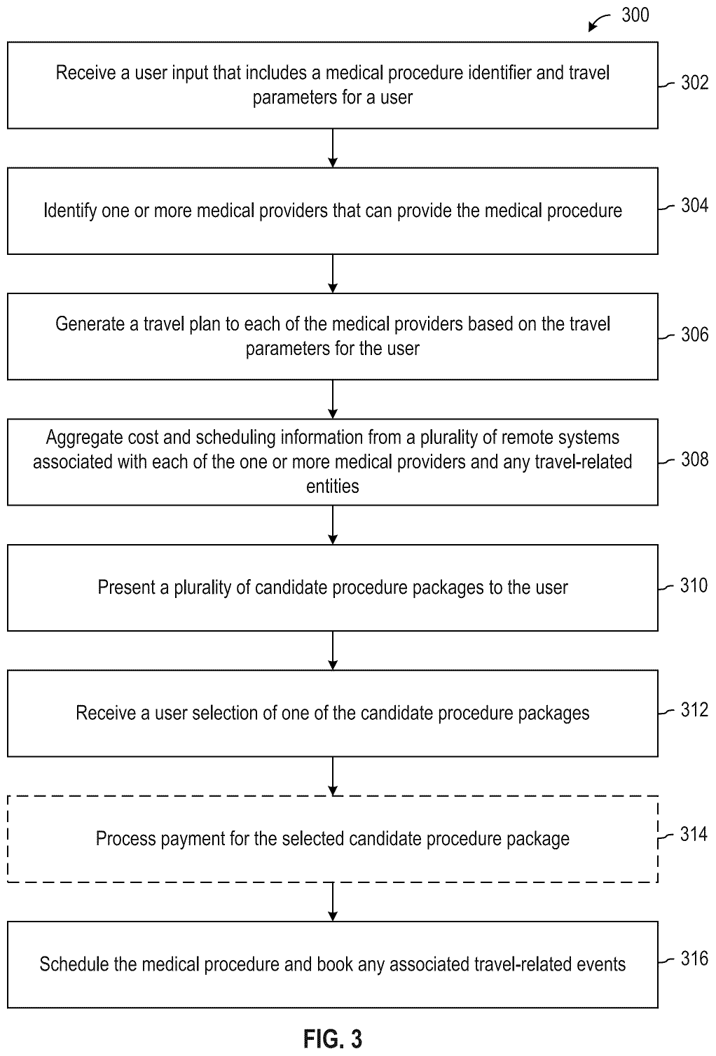

Referring now to FIG. 3, a flow diagram of a process 300 for comparing medical procedure costs is shown, according to some implementations. In particular, process 300 can be used to compare medical procedure costs between multiple different medical providers at various different locations. Notably, process 300 may be implemented to compare pricing for receiving medical treatment domestically or internationally, to include non-medical related expenses such as travel. In some implementations, process 300 is implemented by cost comparison tool 200, as described above. It will be appreciated that certain steps of process 300 may be optional and, in some implementations, process 300 may be implemented using less than all of the steps. It will also be appreciated that the order of steps shown in FIG. 3 is not intended to be limiting.

At step 302, a user input that includes a medical procedure identifier and travel parameters for a user is received. In some implementations, the user input is received from a user device, such as a smartphone, personal computer, or other suitable computing device. In some such implementations, the user may access, via the user device, a software application, website, or other interface that includes one or more fillable fields into which the user can enter the medical procedure identifier and travel parameters. For example, the user may enter the medical procedure identifier and travel parameters into a fillable form on a website, such as by entering text into text entry fields, selecting options from menus, etc. An example of such an interface is described below with respect to FIG. 4. It should be appreciated that the user input of the medical procedure identifier and travel parameters can be received by other means as well. For example, the user could enter a prompt into a chat bot or may send a text message (e.g., “What is the cost of a knee replacement surgery in Spain?”) to cost comparison tool 200.

Generally, the medical procedure identifier is any alphanumeric string that identifies the medical procedure for which the user is comparing costs. For example, the medical procedure identifier can be a name of the medical procedure (e.g., cataract removal, knee or hip replacement, angioplasty, heart bypass surgery, etc.), a billing or procedure code, or the like. Travel parameters can include any data used to identify medical providers that can provide the medical procedure and determine travel-related costs. In some implementations, travel parameters can include one or more of a budget (e.g., for travel alone or combined with the medical procedure), the user's home address, a departure airport or city, preferred travel dates, a preferred departure date, a maximum trip length (e.g., in days), etc.

In some implementations, the user input includes additional information about the user, such as demographic and/or biometric characteristics (e.g., height, weight, age, racial identity, gender identity, etc.), primary medical care provider information (e.g., name, location, contact information), medical insurance information (e.g., group and/or ID number, provider identifier, etc.), medical history, vaccination records, passport status, etc. In some implementations, the user input includes various user preferences such as whether the user prefers to fly or drive, preferred seating position on an airplane (e.g., economy, business, first class), preferred airlines or other travel providers, preferred hospitality providers (e.g., hotel chains), an indication of whether a rental car is needed, and the like. Thus, it will be appreciated that the initial user input may include a variety of data and is not intended to be limited to the data points described herein.

In some implementations, at least a portion of the data provided by the user is used to generate a user profile and/or is stored with a previously generated user profile (e.g., in a database). A user profile is generally a set or directory of stored user data (e.g., user information and settings) associated with a user account. For example, a user profile may be securely accessed by logging in to a user account using a username and/or password. In this way, certain user data (e.g., demographic and/or biometric characteristics, primary medical care provider information, medical insurance information, medical history, vaccination records, passport status, etc.) can be readily accessed without the need for the user to re-input. The user profile may also include a history or “log” of user interactions with cost comparison tool 200, as described in greater detail below.

At step 304, one or more medical providers that can provide the medical procedure are identified. Generally, the one or more medical providers are identified using the medical procedure identifier received at step 302. Optionally, the travel parameters received at step 302 may also be used. In some implementations, the medical procedure identifier is used to query a database or list of medical providers to identify medical providers that provide the medical procedure. For example, cost comparison tool 200 may maintain or access a database of medical providers that includes, for each medical provider, a list of the medical procedures performed. Cost comparison tool 200 may then compare the medical procedure identifier provided by the user with the list of medical procedures performed by each medical provider to identity those medical providers that perform the procedure.

In some implementations, medical providers are identified by transmitting a query to a remote system associated with each medical provider. For example, cost comparison tool 200 may transmit a query (e.g., “Do you perform heart bypass surgery?”), such as via API call, to each medical provider's computing system, and may evaluate the responses (e.g., a yes or no) to generate a list of medical providers that perform the medical procedure. In some implementations, cost comparison tool 200 can include or implement a web crawler that searches the Internet to automatically identify medical providers that perform the medical procedure. It should be appreciated as well that any other method of identifying medical providers that provide a particular medical treatment or procedure may be implemented herein. In many cases, the medical providers identified by cost comparison tool 200 using any of the techniques described herein may have a preexisting agreement with an entity that operates cost comparison tool 200 to be considered in cost comparisons. In other words, only medical providers that have previously agreed to perform certain medical procedures may be identified at step 304, in some implementations.

In some implementations, any medical provider that can provide the user-defined medical treatment is identified at step 304. In other implementations, various user-defined travel parameters may be used to filter or limit the medical providers that are identified. For example, the user may specify certain countries that they will not travel to; thus, any medical providers from the specified countries may be excluded from identification. As another example, the user may specify a maximum travel distance or travel time (e.g., in hours) which may limit the identified medical providers to those within a threshold distance (e.g., by time or miles) of the user. As yet another example, the user's passport status may be used to identify medical providers. For example, an indication that the user's passport is expired or that the user does not have a valid passport may limit the identified medical providers to those in countries that do not require a passport for travel. Or, the user's passport may only be valid in certain countries, in which case the identified medical providers may be limited to those in countries that are valid based on the user's passport. In some implementations, visa requirements of the various countries associated with the medical providers may also be considered when identifying suitable medical providers.

In some implementations, both domestic and international medical providers are identified. In this manner, a user can compare costs of receiving a medical treatment locally with receiving the medical treatment internationally. In some implementations, the user's medical insurance information can be used to identify local (e.g., domestic) medical providers. For example, the identified medical providers may include those medical providers that are “in-network” for the user. In some implementations, domestic medical providers are identified based on their proximity to the user. For example, the user's home address or current location (e.g., determined using the user's device location) can be used to identify those domestic medical providers that are within a threshold distance of the user, such that that the user does not need to travel by airplane to reach the domestic medical providers. To expand on this example, domestic medical providers may be limited to those within driving distance (e.g., 150 miles, 6 hours, etc.) of the user.

At step 306, a travel plan (e.g., itinerary) is generated for each for the one or more identified medical providers. As generally described herein, the one or more identified medical providers generally include multiple international medical providers that are physically located in different countries, and even any domestic medical providers may be disparately located. Thus, each travel plan can include one or more travel events that are required to reach a medical facility associated with a respective one of the one or more medical providers. A “travel event” generally refers to at least a portion of round-trip travel to the medical provider or any event that is included in travel to/from the medical provider. For example, a flight or bus ride may be a travel event. Travel events can also include shuttle rides, car rentals, stays at hospitality facilities (e.g., hotels, motels, etc.), and the like. Thus, as part of generating the travel plan, cost comparison tool 200 may identify various travel entities (e.g., airlines, train lines, shuttle companies, hotels, motels, etc.) that can be used to travel to/from the destination (e.g., a city or medical facility associated with each identified medical provider).

Say, for example, that one of the identified medical providers is located in Istanbul, Turkey, while the user (e.g., of cost comparison tool 200) is located in New York city. The generated travel plan for the identified medical provide may therefore include round-trip airfare between one of New York's airports and one of Istanbul's airports. The generated travel plan may also include shuttles, buses, or public transport to/from each airport, such as a shuttle from the Istanbul airport to a local hotel. To this point, as part of generating the travel plan, one or more hotels within a threshold distance (e.g., 10 miles) of either the medical facility associated with the medical provider, or the destination airport, may be identified.

At step 308, cost and scheduling information is aggregated from a plurality of remote systems associated with each of the one or more medical providers and any travel entities. Put another way, cost comparison tool 200 is generally configured to collect and combine cost and scheduling data from each identified medical provider with cost and scheduling data for each travel entity (e.g., airline, hotel, etc.) identified in an associated travel plan. Taking the previous example one step further, this may include: i) collecting cost and scheduling data from the medical provider in Istanbul, ii) collecting cost and scheduling data for round-trip airfare to/from the Istanbul airport (e.g., from various airlines), iii) collecting cost and scheduling data for a hotel stay in Istanbul (e.g., from multiple hotel chains), and iv) combining the cost and scheduling data for the medical provider and travel entities to generate a total price for the procedure and travel package, herein referred to as a “candidate procedure package.”

The aggregation of cost and scheduling information from remote systems is generally described above with respect to FIG. 2; thus, for the sake of brevity, the details of this aggregation process are not repeated here. However, at a high level, cost comparison tool 200 may aggregate cost and scheduling information by first retrieving or requesting cost and scheduling data from each medical provider and travel entity (e.g., based on the medical providers and travel entities identified at steps 304 and 306). For example, in some implementations, cost comparison tool 200 may transmit queries (e.g., as API calls) to remote systems associated with each medical provider and travel entity to request current cost and scheduling information. Assuming that the requested cost and scheduling data is available, each remote system then replies with the requested information. For example, each remote system may return the requested information via API call to cost comparison tool 200. In some implementations, cost comparison tool 200 is able to directly access one or more remote systems to retrieve cost and/or scheduling data.

In some instances, a remote source may return a message indicating that the requested cost and/or scheduling information is not available. Alternatively, cost comparison tool 200 may receive only one of cost information or scheduling information. In some such implementations, cost comparison tool 200 may be configured to estimate or predict the unavailable cost or scheduling information. For example, if cost information is not available from one or more medical provider, cost comparison tool 200 may either remove the medical provider from consideration (e.g., assuming other medical providers are available) or may estimate cost information based on historical data. In some implementations, cost comparison tool 200 maintains and/or has access to a database of costs for flights, medical procedures, etc., and can therefore retrieve past cost data to estimate current costs. For example, cost comparison tool 200 may determine an average cost for a medical procedure (e.g., by provider, by city, by country) based on historical pricing data.

Once the cost and scheduling information is obtained, cost comparison tool 200 is configured to generate a plurality of candidate procedure packages. Each candidate procedure package may include a total cost for the medical procedure and any travel-related expenses based on the aggregate cost data from the plurality of sources. For example, each candidate procedure package may include a total cost for the medical procedure, round-trip travel, hotel stays, etc. Additionally, each candidate procedure package generally includes a schedule or itinerary based on the aggregate scheduling data. For example, each candidate procedure package may indicate a departure and return date based on flight information. In some implementations, multiple candidate procedure packages are generated for each medical provider and/or for each location (e.g., each country or city). For example, each candidate procedure package for a first medical provider may include different round-trip travel options (e.g., different airlines, different transportation methods, different hotels), which may all affect scheduling and/or cost. As another example, multiple candidate procedure packages can be generated for a particular country (e.g., Canada) which include different medical provider and travel options.

To further illustrate this point, consider a user that implements process 300 (e.g., via cost comparison tool 200) to compare pricing for a root canal. Using the user provided medical procedure identifier and travel parameters, cost comparison tool 200 may identify multiple different medical providers in multiple different countries that can provide a root canal (optionally, within the user's requested time frame). Cost comparison tool 200 can then determine a travel plan to each different medical provider and can aggregate cost and scheduling information for each medical provider and multiple travel-related entities (e.g., airlines, hotels, etc.) to generate a plurality of candidate procedure packages, each with a unique cost and schedule. For example, a first candidate procedure package may include a total cost of receiving a root canal at a first medical provider in Canada, to include round-trip travel via a first airline; a second candidate procedure package may include a total cost of receiving a root canal at the first medical provider in Canada, to include round-trip travel via a second airline; and a third procedure package may include a total cost of receiving a root canal at a second medical provider in Canada, to include round-trip travel via the first airline.

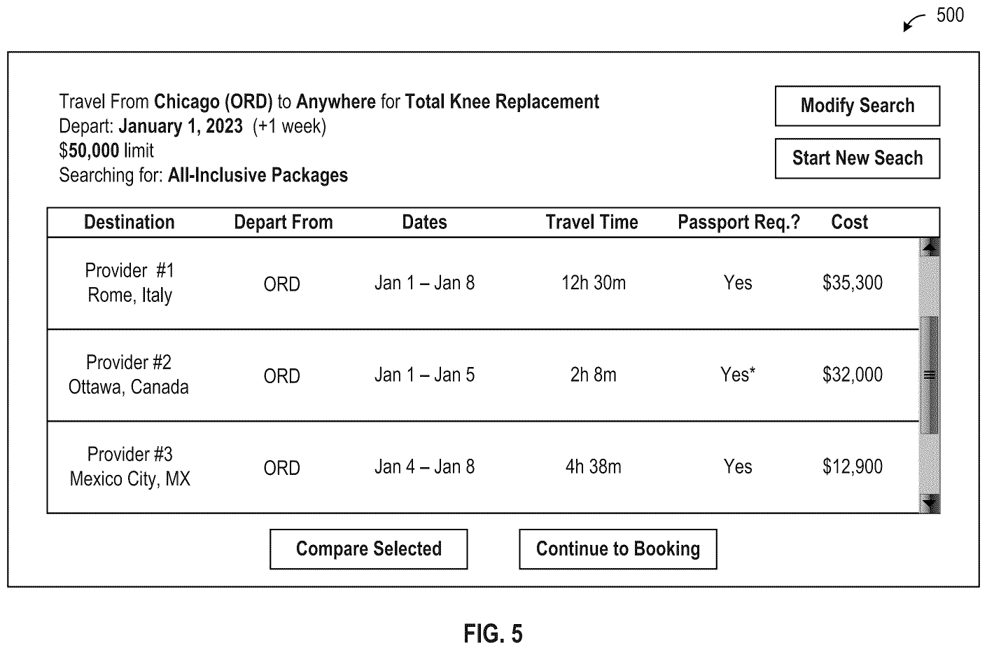

At step 310, the plurality of generated candidate procedure packages are presented to the user. In some implementations, cost comparison tool 200 generates a user interface to present the candidate procedure packages. In some such implementations, the user interface is presented via a display of cost comparison tool 200 or user device 130. For example, cost comparison tool 200 may transmit the user interface to user device 130 for display. An example of such a user interface is shown in FIG. 5. It will be appreciated, however, that the candidate procedure packages can presented to a user by any other suitable method. For example, the candidate procedure packages can be sent to a user via email or by text message. Generally, each candidate procedure package that is presented indicates at least an identifier for the associated medical provider or an identifier for a location associated with the medical provider (e.g., a city), a departure date for traveling to the medical provider to receive treatment, and a total cost of the package, although additional information can also be presented.

At step 312, a user selection of one of the plurality of candidate procedure packages is received. In some implementations, the user selection is received as an input to a user interface of user device 130. For example, the user may select one of the candidate procedure packages by tapping or clicking on the displayed candidate procedure package. In other implementations, the user selection can be received via text message, email, or chat. In some implementations, the user may use a digital assistant to select a package, such as by verbally instructing the digital assistant to select the package.

At step 314, payment is optionally processed (e.g., adjudicated) for the selected candidate procedure package. In some implementations, cost comparison tool 200 is configured to transmit payment to each of the remote systems associated with the medical provider and travel-related entities defined in the select package. For example, cost comparison tool 200 may transmit payment for the medical procedure to the medical provider and may transmit payment to an airline to pay for tickets. In some implementations, cost comparison tool 200 is further configured to collect payment from the user. For example, cost comparison tool 200 may prompt the user to enter bank or credit card details and may accordingly withdraw funds from the user's account to pay for the selected package. In some implementations, cost comparison tool 200 may prompt the user for a single payment that matches the cost of the package and may distribute the payment to each respective entity (e.g., the medical provider, an airline, etc.) accordingly. In some implementations, cost comparison tool 200 can adjudicate certain payments after the medical procedure or travel is completed. In some such implementations, cost comparison tool 200 may receive and hold user funds for later distribution. For example, in some cases, payment for the medical procedure is due after the procedure is complete, whereas payment for travel-related expenses is due at the time of booking. In such cases, cost comparison tool 200 may transmit payments to each travel-related entity while holding funds to pay for the medical procedure at a later date.

At step 316, the medical procedure is scheduled, and any travel-related events are booked. In some implementations, cost comparison tool 200 is configured to automatically schedule/book each portion of the selected candidate procedure package without additional user input. For example, cost comparison tool 200 may transmit requests for each remote system to schedule a corresponding event (e.g., the procedure, travel, etc.). In some implementations, cost comparison tool 200 transmits requests via API call to each respective remote systems API 112-116. In some implementations, cost comparison tool 200 automatically schedules/books each portion of the selected candidate procedure package responsive to the user submitting payment for the package. In some implementations, one or more portions of the package may not be automatically scheduled/booked, in which case cost comparison tool 200 may automatically open or navigate to a website or scheduling program for each respective entity. For example, plane tickets may be automatically booked without user input, but the medical procedure may require additional detail and/or the medical provider may not have the capability of automatic scheduling; thus, cost comparison tool 200 may automatically navigate the user to the medical provider's website for booking.

In some implementations, cost comparison tool 200 can make phone calls to schedule portions of the package. For example, cost comparison tool 200 may use a speech recognition model to automatically call certain entities to schedule/book a portion of the selected candidate procedure package. In some implementations, cost comparison tool 200 is configured to automatically update the user's calendar when a portion of the package, or the entire package, is scheduled/booked. In some implementations, cost comparison tool 200 can send a message or alert to the user with a status of the scheduling/booking. For example, cost comparison tool 200 may send an email when booking is complete for all portions of the selected package. In another example, cost comparison tool 200 can generate and send an alert if one or more portions of the package cannot be scheduled/booked. In some implementations, cost comparison tool 200 can prompt the user to provide additional information, as needed, for booking/scheduling. For example, cost comparison tool 200 may prompt the user to enter details such as their date of birth or known traveler ID number for booking airfare.

Generally, cost comparison tool 200 is further configured to record and/or generate a log of each transaction (e.g., comparison). For example, each time a user interacts with cost comparison tool 200, and/or each time process 300 is executed, cost comparison tool 200 may generate a record user inputs and/or other actions. For example, cost comparison tool 200 may generate a record of aggregated cost and scheduling data to build or maintain a database of cost and scheduling information. In some implementations, transaction data is used to generate a log for the user (e.g., stored with the user profile) for future reference. For example, cost comparison tool 200 may maintain a log of past procedures or trips for a user. As another example, cost comparison tool 200 may maintain a record of a user's past searches (e.g., comparisons).

Referring now to FIG. 4, an example user interface 400 for entering a medical procedure identifier and travel parameters is shown, according to some implementations. In some implementations, user interface 400 is an example of a graphical user interface (GUI) generated by user interface generator 220 and displayed via remote device 130 and/or cost comparison tool 200. For example, user interface 400 may be an example web page displayed to a user upon navigating to a website associated with cost comparison tool 200. In other words, user interface 400 may be a “front end” interface of cost comparison tool 200. In any case, user interface 400 is generally shown to include a plurality of fields in which a user can enter details of the medical procedure being searched/compared.

In the example shown, user interface 400 includes a first text field for entering a procedure identifier (e.g., a procedure name). In some implementations, the procedure identifier may be all that is needed to initiate a search, in which case cost comparison tool 400 may generate candidate procedure packages for any medical provider (e.g., in the world) that provides the identified service. In some implementations, user interface 400 also includes fields for entering a departure city and/or airport (or the user's current address), preferred travel dates (e.g., departure and/or return date, maximum or preferred trip length, etc.), and budget. In some implementations, user interface 400 may also include fields for selecting a breadth of the search; for example, whether the user is searching for all-inclusive packages (e.g., which include the procedure and round-trip travel), the procedure only, or the procedure and round-trip airfare (e.g., not include hotel stays, etc.).

It should be appreciated that user interface 400 is not limited to only those fields/parameters shown. Rather, user interface 400 may include fewer or additional fields for entering other search parameters. Further, it will be appreciated that any suitable graphical elements can be used. For example, user interface 400 may include one or more of text entry fields, menus (e.g., drop-down menus), icons, buttons, and the like. In some implementations, user interface 400 includes a “Clear” button which can be selected to clear the fields from any user-populated data. In some implementations, user interface 400 includes a “Search” button which initiates a search/comparison using the user-populated data. In some implementations, responsive to the user entering data into one or more field and selecting the “Search” button, the interface shown in FIG. 5 may be presented. To this point, selecting the “Search” button generally initiates process 300, described above.

Referring now to FIG. 5, an example user interface 500 for presenting a plurality of candidate procedure packages is shown, according to some implementations. Like user interface 400 described above, user interface 500 is generally an example of a GUI generated by user interface generator 220 and displayed via remote device 130 and/or cost comparison tool 200. As shown, user interface 500 includes a list of candidate procedure packages and provides various details about each package. Specifically, in this example, each package indicates the medical provider, a destination city, a departure airport identifier, travel dates, total travel time, passport requirements, and a package cost.

In the example shown, the user is search for/comparing costs of a “Total Knee Replacement” surgery. Travel-related details (e.g., entered by the user) include the departure airport (“Chicago (ORD)), preferred departure date (“Jan. 1, 2023″ or within one week of January 1^{st}), budget (“$50,000), and search parameters (“All-Inclusive Packages”). Based on these details, multiple candidate procedure packages are generated and displayed. The first example package shown is for “Provider #1” in Rome, Italy, at a total cost of $35,300. This package includes round-trip flight from Chicago to Rome, with departure on January 1^{st} and return on January 8^{th}.

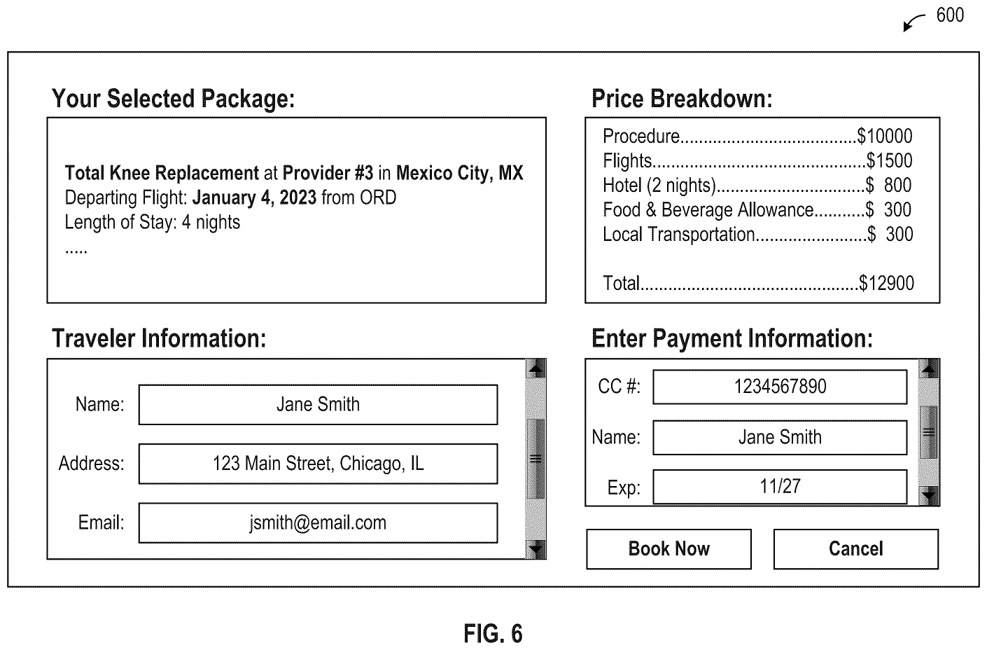

In some implementations, user interface 500 includes multiple graphical elements (e.g., buttons) for modifying search parameters or starting a new search. For example, user interface 500 is shown to include a “Modify Search” button which all the user to modify one or more parameters (e.g., departure airport, budget, etc.). In some implementations, selecting the “Modify Search” button may return the user to user interface 400. User interface 500 also includes a “Start New Search” button returns the user to user interface 400 for starting a new search. In some implementations, a user can select more than one package and then a “Compare Selected” button in order to compare additional details of the packages (e.g., which may not be shown in user interface 500). Once a single package is selected, the user may select a “Continue to Booking” button to proceed to a booking/scheduling and/or payment page (e.g., as in FIG. 6, described below).

Referring now to FIG. 6, an example user interface 600 for booking a candidate procedure package is shown, according to some implementations. As with the above-described user interfaces, user interface 600 is generally an example of a GUI generated by user interface generator 220 and displayed via remote device 130 and/or cost comparison tool 200. In some implementations, user interface 600 is a “checkout” page for completing payment and/or booking of a procedure package. In this example, user interface 600 is shown to include a summary of the selected package (e.g., “Total Knee Replacement at Provider #3 in Mexico City, MX”). User interface 600 may also include a field that displays a price breakdown of the package, such that the user can view the cost of each individual portion of the selected package.

In some implementations, user interface 600 includes a “Traveler Information” field with includes one or more text entry boxes in which a user can enter personal details, such as name, address, email, phone number, birthdate, etc. Finally, user interface 600 is shown to include an optional payment entry field, which includes multiple text entry boxes or menus in which a user can enter payment details. In this example, the user is paying by credit card and has begun entering credit card details (e.g., number, expiration date, etc.). In some implementations, the payment entry field includes an embedded widget, or simply acts as an interface, for a separate banking or payment processing application. For example, payment information may be entered into the fields of the payment entry field and then processed via a payment processor's API. Once all of the information in user interface 600 is entered and/or confirmed, the user may select a “Book Now” button to proceed with payment and/or booking/scheduling the package. Optionally, the user can choose to return back to user interface 400 or 500 by selecting a “Cancel” button, which may also erase the data entered into any of the fields of user interface 600.

### Configuration of Certain Implementations

The construction and arrangement of the systems and methods as shown in the various exemplary implementations are illustrative only. Although only a few implementations have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the exemplary implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

What is claimed is:

1. A method comprising:
receiving a first user input that includes a medical procedure identifier and travel parameters for a user;
identifying one or more medical providers that can provide a medical procedure associated with the medical procedure identifier;
generating a plurality of candidate procedure packages for the medical procedure by aggregating cost and scheduling information from a plurality of remote sources associated with each of the one or more medical providers and one or more travel-related entities, wherein the one or more travel-related entities are identified based on the travel parameters, and wherein each of the plurality of candidate procedure packages indicates: i) a cost for performing the medical procedure at a medical facility associated with a respective one of the one or more medical providers, and ii) a cost for round-trip travel to/from the medical facility;
presenting the plurality of candidate procedure packages to the user via a user interface;
receiving a second user input selecting one of the plurality of candidate procedure packages; and
scheduling the medical procedure and booking round-trip travel through a respective subset of the plurality of remote sources.

2. The method of claim 1, wherein the travel parameters include at least one of budget, a departure city, a home address of the user, preferred travel dates, and a preferred departure date.

3. The method of claim 1, wherein each of the plurality of candidate procedure packages further includes a cost for a stay at a hospitality facility within a threshold distance of the medical facility.

4. The method of claim 1, wherein generating the plurality of candidate procedure packages comprises:
transmitting one or more application programming interface (API) calls to each of the plurality of remote sources, wherein the one or more API calls cause the plurality of remote sources to return individual cost and scheduling information; and
receiving the individual cost and scheduling information from each of the plurality of remote sources; and
aggregating the individual cost and scheduling information from each of the plurality of remote sources into the plurality of candidate procedure packages.

5. The method of claim 1, wherein scheduling the medical procedure and booking the round-trip travel comprises:
transmitting one or more application programming interface (API) calls to each of the plurality of remote sources, wherein the one or more API calls cause respective ones of the plurality of remote sources to schedule the medical procedure or book at least a portion of the round-trip travel; and
receiving, from each of the plurality of remote sources, i) a confirmation that the medical procedure has been booked or that the at least a portion of the round-trip travel has been booked, and ii) a final cost associated with the medical procedure or the at least a portion of the round-trip travel.

6. The method of claim 1, further comprising adjudicating payment for the medical procedure and round-trip travel.

7. The method of claim 6, wherein the medical procedure is scheduled and the round-trip travel is booked automatically after the user submits payment for the medical procedure and the round-trip travel.

8. The method of claim 1, wherein presenting the plurality of candidate procedure packages to the user comprises displaying, for each of the plurality of candidate procedure packages, an identifier for the medical facility, a destination city name, travel dates, and a total cost of the candidate procedure package.

9. The method of claim 1, wherein the first user input further includes insurance information for the user, and wherein the plurality of candidate procedure packages includes at least one candidate procedure package for a domestic medical provider covered by the user's insurance.

10. The method of claim 1, wherein at least one or more medical providers is located internationally with respect to a home country of the user.

11. A system comprising:
a processor; and
memory having instructions stored thereon that, when executed by the processor, cause the system to:
receive a first user input that includes a medical procedure identifier and travel parameters for a user;
identify one or more medical providers that can provide a medical procedure associated with the medical procedure identifier;
generate a plurality of candidate procedure packages for the medical procedure by aggregating cost and scheduling information from a plurality of remote sources associated with each of the one or more medical providers and one or more travel-related entities, wherein the one or more travel-related entities are identified based on the travel parameters, and wherein each of the plurality of candidate procedure packages indicates: i) a cost for performing the medical procedure at a medical facility associated with a respective one of the one or more medical providers, and ii) a cost for round-trip travel to/from the medical facility;
present the plurality of candidate procedure packages to the user via a user interface;
receive a second user input selecting one of the plurality of candidate procedure packages; and
schedule the medical procedure and booking round-trip travel through a respective subset of the plurality of remote sources.

12. The system of claim 11, wherein the travel parameters include at least one of budget, a departure city, a home address of the user, preferred travel dates, and a preferred departure date.

13. The system of claim 11, wherein each of the plurality of candidate procedure packages further includes a cost for a stay at a hospitality facility within a threshold distance of the medical facility.

14. The system of claim 11, wherein to generate the plurality of candidate procedure packages, the instructions further cause the system to:
transmit one or more application programming interface (API) calls to each of the plurality of remote sources, wherein the one or more API calls cause the plurality of remote sources to return individual cost and scheduling information; and
receive the individual cost and scheduling information from each of the plurality of remote sources; and
aggregate the individual cost and scheduling information from each of the plurality of remote sources into the plurality of candidate procedure packages.

15. The system of claim 11, wherein to schedule the medical procedure and book the round-trip travel, the instructions further cause the system to:
transmit one or more application programming interface (API) calls to each of the plurality of remote sources, wherein the one or more API calls cause respective ones of the plurality of remote sources to schedule the medical procedure or book at least a portion of the round-trip travel; and
receive, from each of the plurality of remote sources, i) a confirmation that the medical procedure has been booked or that the at least a portion of the round-trip travel has been booked, and ii) a final cost associated with the medical procedure or the at least a portion of the round-trip travel.

16. The system of claim 11, wherein the instructions further cause the system to adjudicate payment for the medical procedure and round-trip travel.

17. The system of claim 15, wherein the medical procedure is scheduled and the round-trip travel is booked automatically after the user submits payment for the medical procedure and the round-trip travel.

18. The system of claim 11, wherein to present the plurality of candidate procedure packages to the user, the instructions further cause the system to:
display, for each of the plurality of candidate procedure packages, an identifier for the medical facility, a destination city name, travel dates, and a total cost of the candidate procedure package.

19. The system of claim 11, wherein the first user input further includes insurance information for the user, and wherein the plurality of candidate procedure packages includes at least one candidate procedure package for a domestic medical provider covered by the user's insurance.

20. A non-transitory computer readable medium having instructions stored thereon that, when executed by a processor, cause a device to:
receive a first user input that includes a medical procedure identifier and travel parameters for a user;
identify one or more medical providers that can provide a medical procedure associated with the medical procedure identifier;
generate a plurality of candidate procedure packages for the medical procedure by aggregating cost and scheduling information from a plurality of remote sources associated with each of the one or more medical providers and one or more travel-related entities, wherein the one or more travel-related entities are identified based on the travel parameters, and wherein each of the plurality of candidate procedure packages indicates: i) a cost for performing the medical procedure at a medical facility associated with a respective one of the one or more medical providers, and ii) a cost for round-trip travel to/from the medical facility;
present the plurality of candidate procedure packages to the user via a user interface;
receive a second user input selecting one of the plurality of candidate procedure packages; and
schedule the medical procedure and booking round-trip travel through a respective subset of the plurality of remote sources.
